(19) United States
(12) Patent Application Publication
(10) Pub. No.: US 2026/0103441 A1
(43) Pub. Date: Apr. 16, 2026

(54) COCRYSTAL OF LUTEIN AND ADIPIC ACID, PREPARATION METHOD THEREFOR AND USE THEREOF

(71) Applicant: Cocrystal Health Industry (Zhejiang) Co., Ltd., Jiashan (CN)

(72) Inventors: Xuefeng MEI, Jiashan (CN); Chenxuan ZHENG, Jiashan (CN); Qi ZHANG, Jiashan (CN); Junjie BAO, Jiashan (CN); Da LEI, Jiashan (CN)

(21) Appl. No.: 18/917,697

(22) Filed: Oct. 16, 2024

(51) Int. Cl. C07C 403/24; A61K 31/047; A61K 47/12; C07C 51/43; C07C 55/14

(52) U.S. Cl. CPC C07C 403/24; A61K 31/047; A61K 47/12; C07C 51/43; C07C 55/14; C07B 2200/13

(57) ABSTRACT

The present invention discloses a cocrystal of lutein and adipic acid, a preparation process and a use thereof, wherein the stoichiometric ratio of lutein to adipic acid in the cocrystal is 1:1. The preparation process disclosed in the present invention is simple to operate, and has the advantages of being low cost, environmentally friendly, easy to control and well reproducible. In addition, the cocrystal disclosed in the present invention has more excellent chemical stability, greatly improves the convenience of the application of lutein, saves costs in storage, transportation and use, and can further broaden the application range of lutein.

### FIELD OF THE INVENTION

The present invention relates to the lutein technical field, and specifically, to a cocrystal of lutein and adipic acid, a preparation process and a use thereof.

### BACKGROUND OF THE INVENTION

Lutein, also known as “plant lutein”, is a natural pigment widely found in vegetables, flowers, fruits and certain algaes. Lutein is the main pigment constituting the macular area of the human retina. It can filter blue light and prevent retinal damage, and has a positive effect on maintaining eye health and protecting vision. In recent years, a large number of studies have shown that lutein also has antioxidant, free radical scavenging, anti-cancer and anti-tumor effects. Lutein is a natural carotenoid with bright color, strong coloring power, good antioxidant properties, safety and non-toxicity, and it has rich nutritional value and is widely used in food, health products, cosmetics, medicine, tobacco, animal feed, etc. In 1995, the U.S. FDA approved lutein as a food nutritional supplement and colorant. Lutein is also listed as a permitted food additive in GB2760-1996 in China.

However, lutein is a fat-soluble pigment with high unsaturation and easy oxidation, which limits the use of lutein as a nutritional enhancer and natural pigment in food. Studies have shown that lutein is very sensitive to factors such as temperature, light, oxygen, reducing agents and oxidants, so lutein crystals should be stored at low temperature, away from light and in a vacuum. The main process currently used to improve the stability of lutein is microencapsulation embedding technique. However, the lutein content in the embedded powder is very low (the lutein content of commercially available embedded powder is generally 5% and 10%). Meanwhile, the embedding process is relatively complicated and requires high-energy consumption and high-cost production processes such as high-temperature emulsification and spray drying. Therefore, it is a goal that people continue to pursue to obtain a lutein product that is easy to use, has a high content, is chemically stable, has a simple process and is low in cost.

### SUMMARY OF THE INVENTION

In order to improve the stability of lutein products, the present inventor attempts to form cocrystals of lutein with lots of compounds through a large number of experiments. It has been found that by adding edible adipic acid as a ligand, a stable cocrystal can be formed, thereby changing the intermolecular interaction and spatial arrangement of lutein molecules at the molecular level, enhancing the stability of lutein molecules to oxygen and temperature, and improving its chemical stability and broadening its application field. Therefore, the solution of preparing a cocrystal with lutein and adipic acid is determined to solve the above technical problems, thereby completing the present invention.

A Lutein cocrystal with better stability can further broaden the application range of lutein. Therefore, the cocrystal of lutein and adipic acid described in the present invention has a strong practical application value.

One of the purposes of the present invention is to provide a cocrystal of lutein and adipic acid.

The second purpose of the present invention is to provide a process for preparing the cocrystal of lutein and adipic acid.

The third purpose of the present invention is to provide a composition comprising the above-mentioned cocrystal of lutein and adipic acid.

The fourth object of the present invention is to provide a use of the cocrystal of lutein and adipic acid or the composition in preparing a product, wherein the product is selected from health products, food, cosmetics, medicines, pharmaceutical excipients and feed. According to one aspect of the present invention, a cocrystal of lutein and adipic acid is provided, wherein the stoichiometric ratio of lutein to adipic acid in the cocrystal is 1:1. In particular, the cocrystal of lutein and adipic acid is characterized by an X-ray powder diffraction pattern comprising characteristic peaks at 2θ of 3.4°±0.2°, 12.2°±0.2°, 13.6°±0.2°, 15.7°±0.2°, 16.6°±0.2°, and 17.8°±0.2°; more particularly, the cocrystal of lutein and adipic acid is characterized by an X-ray powder diffraction pattern comprising characteristic peaks at 2θ of 3.4°±0.2°, 12.2°±0.2°, 13.6°±0.2°, 15.7°±0.2°, 16.6°±0.2°, 17.8°±0.2°, 18.2±0.2°, and 21.5±0.2°; particularly, the cocrystal of lutein and adipic acid is characterized by an X-ray powder diffraction pattern substantially as shown in FIG. 1.

In particular, the cocrystal of lutein and adipic acid is characterized by a differential scanning calorimetry analysis curve comprising a characteristic endothermic peak at about 187±2° C., when the temperature is increased at a rate of 10° C./min as measured by differential scanning calorimetry; preferably, it is characterized by a differential scanning calorimetry analysis curve substantially as shown in FIG. 2.

In particular, the cocrystal of lutein and adipic acid is characterized by an infrared spectrum comprising characteristic peaks at about 3446 cm⁻¹, 1723 cm⁻¹, and 963 cm⁻¹; in particular, the infrared spectrum further comprises characteristic peaks at 3023 cm⁻¹, 2957 cm⁻¹, 2865 cm⁻¹, 1567 cm⁻¹, 1411 cm⁻¹, 12778 cm⁻¹, and 1190 cm⁻¹; preferably, it is characterized by an infrared spectrum diagram substantially as shown in FIG. 3.

According to the second aspect of the present invention, the present invention provides a process for preparing the cocrystal of lutein and adipic acid, which is one of the following processes:
Process 1: recrystallizing lutein and adipic acid in a molar ratio of 1:1 in a solvent, and drying the resultant precipitate to obtain the cocrystal of lutein and adipic acid;
Process 2: ball-milling lutein and adipic acid in a molar ratio of 1:1 in a solvent for 10 minutes or more, and then drying the obtained solid to obtain the cocrystal of lutein and adipic acid.

In particular, in process 1, lutein and adipic acid may be stirred and suspended in a solvent, for example, at 10-40° C., for example, stirred for 5-30 hours, and the resulting precipitate (for example, obtained by filtration) is dried to obtain a cocrystal of lutein and adipic acid.

In particular, in process 2, lutein and adipic acid may be ball-milled in a solvent for 10 minutes or more, for example, for 30 minutes to 2 hours, and then the obtained solid is dried (for example, dried in a vacuum, for example, dried at room temperature for 10-15 hours) to obtain a cocrystal of lutein and adipic acid.

The above solvent is selected from the solvents that have a certain solubility to the raw materials and do not cause deterioration of the raw materials. Preferably, the solvent is one or more selected from the group consisting of water, alcohols, ketones, esters, alkanes, aromatic hydrocarbons and halogenated alkanes; more preferably, the solvent is one or more selected from the group consisting of methanol, ethanol, isopropanol, ethyl acetate, isopropyl acetate, acetone, methyl ethyl ketone, methyl tert-butyl ether, n-hexane, and n-heptane.

The preparation process involved in the present invention is simple to operate, the crystallization process is easy to control, the crystallinity is high, and the reproducibility is good, and the cocrystal of lutein and adipic acid may be stably obtained.

In a third aspect, the present invention provides a lutein composition comprising the above-mentioned cocrystal of lutein and adipic acid.

In some embodiments, the lutein composition may contain an excess amount of adipic acid or an excess amount of lutein, and other excipients in addition to the cocrystal of lutein and adipic acid according to the present invention. That is to say, in the raw materials of the lutein composition, the molar ratio of lutein to adipic acid is not particularly limited, as long as the lutein composition contains the above-mentioned cocrystal of lutein and adipic acid, or its raw materials can be used to prepare the above-mentioned cocrystal of lutein and adipic acid. For example, the stoichiometric ratio of lutein to adipic acid in the lutein composition may be 10:1 to 1:10, wherein a part of the components exist in the form of cocrystal of lutein and adipic acid, and another part of the components exist in free form. It is preferred that all the lutein forms the cocrystal to overcome the defect of poor stability of lutein; or, in the lutein composition, the composition is substantially composed of cocrystal of lutein and adipic acid. The other excipients are not particularly limited and may vary according to the purpose of application. For example, when applied to drugs, they may be pharmaceutically acceptable excipients; when applied to health products, they may be acceptable excipients for health products; when applied to food, they may be acceptable excipients for food; when applied to cosmetics, they may be acceptable excipients for cosmetics; when applied to feed, they may be acceptable excipients for feed.

In some embodiments, the stoichiometric ratio of lutein to adipic acid in the lutein composition may be 1:10, 1:5, 1:2, 2:1, 5:1, or 10:1.

When the stoichiometric ratio of lutein to adipic acid in the lutein composition is 1:2, the composition is characterized by an X-ray powder diffraction pattern comprising characteristic peaks at 2θ of approximately 3.4°±0.2°, 12.2°±0.2°, 13.5°±0.2°, 15.7°±0.2°, 16.7°±0.2°, and 17.8°±0.2°. It has characteristic peaks of the cocrystal of the lutein and adipic acid according to the present invention, thereby confirming that the composition contains the cocrystal of the present invention.

The lutein composition with a stoichiometric ratio of lutein to adipic acid of 1:2 has an X-ray powder diffraction pattern substantially as shown in FIG. 4.

In some other embodiments, the stoichiometric ratio of lutein to adipic acid in the lutein composition is 2:1.

The lutein composition with a stoichiometric ratio of lutein to adipic acid of 2:1 is characterized by an X-ray powder diffraction pattern comprising characteristic peaks at 2θ of approximately 3.4°±0.2°, 12.3°±0.2°, 13.5°±0.2°, 15.8°±0.2°, 16.7°±0.2°, and 17.7°±0.2°. It has characteristic peaks of the cocrystal of lutein and adipic acid according to the present invention, thereby confirming that the cocrystal in the composition is consistent with the crystal form of the cocrystal of the present invention.

The lutein composition with a stoichiometric ratio of lutein to adipic acid of 1:2 has an X-ray powder diffraction pattern substantially as shown in FIG. 5.

In some other embodiments, the stoichiometric ratio of lutein to adipic acid in the lutein composition is 1:5.

The lutein composition with a stoichiometric ratio of lutein to adipic acid of 2:1 is characterized by an X-ray powder diffraction pattern comprising characteristic peaks at 2θ of approximately 3.3°±0.2°, 12.1°±0.2°, 13.5°±0.2°, 15.8°±0.2°, 16.6°±0.2°, and 17.7°±0.2°. It has characteristic peaks of the cocrystal of lutein and adipic acid according to the present invention, thereby confirming that the cocrystal in the composition is consistent with the crystal form of the cocrystal of the present invention.

The lutein composition with a stoichiometric ratio of lutein to adipic acid of 1:5 has an X-ray powder diffraction pattern substantially as shown in FIG. 6.

In some other embodiments, the stoichiometric ratio of lutein to adipic acid in the lutein composition is 5:1.

The lutein composition with a stoichiometric ratio of lutein to adipic acid of 5:1 is characterized by an X-ray powder diffraction pattern comprising characteristic peaks at 2θ of approximately 3.4°±0.2°, 12.2°±0.2°, 13.5°±0.2°, 15.8°±0.2°, 16.5°±0.2°, and 17.5°±0.2°. It has characteristic peaks of the cocrystal of lutein and adipic acid according to the present invention, thereby confirming that the cocrystal in the composition is consistent with the crystal form of the cocrystal of the present invention.

The lutein composition with a stoichiometric ratio of lutein to adipic acid of 5:1 has an X-ray powder diffraction pattern substantially as shown in FIG. 7.

In some other embodiments, the stoichiometric ratio of lutein to adipic acid in the lutein composition is 1:10.

The lutein composition with a stoichiometric ratio of lutein to adipic acid of 1:10 is characterized by an X-ray powder diffraction pattern comprising characteristic peaks at 2θ of approximately 3.3°±0.2°, 12.0°±0.2°, 13.5°±0.2°, 15.8°±0.2°, 16.6°±0.2°, and 17.7°±0.2°. It has characteristic peaks of the cocrystal of lutein and adipic acid according to the present invention, thereby confirming that the cocrystal in the composition is consistent with the crystal form of the cocrystal of the present invention.

The lutein composition with a stoichiometric ratio of lutein to adipic acid of 1:10 has an X-ray powder diffraction pattern substantially as shown in FIG. 8.

In some other embodiments, the stoichiometric ratio of lutein to adipic acid in the lutein composition is 10:1.

The lutein composition with a stoichiometric ratio of lutein to adipic acid of 10:1 is characterized by an X-ray powder diffraction pattern comprising characteristic peaks at 2θ of approximately 3.3°±0.2°, 12.2°±0.2°, 13.5°±0.2°, 15.6°±0.2°, 16.5°±0.2°, and 17.5°±0.2°. It has characteristic peaks of the cocrystal of lutein and adipic acid according to the present invention, thereby confirming that the cocrystal in the composition is consistent with the crystal form of the cocrystal of the present invention.

The lutein composition with a stoichiometric ratio of lutein to adipic acid of 10:1 has an X-ray powder diffraction pattern substantially as shown in FIG. 9.

In another aspect, the present invention provides a lutein product comprising the cocrystal of lutein and adipic acid or the above-mentioned lutein composition, wherein the product is selected from health products, foods, cosmetics, medicines, pharmaceutical excipients, and feeds.

On the other hand, the present invention provides a use of the cocrystal of lutein and adipic acid or the above-mentioned lutein composition in the preparation of a lutein product, and the product is selected from health products, foods, cosmetics, medicines, pharmaceutical excipients and feeds.

The product may also contain other suitable raw materials required for the product, for example, for foods, the product may contain main food ingredients and edible food additives acceptable to foods, such as sweeteners, flavors, preservatives, fragrances, colorants, etc.; for cosmetics, the product may contain main cosmetic ingredients and additives acceptable to cosmetics, such as solvents, fragrances, preservatives, flavors, colorants, etc.; for medicines, the product may contain pharmaceutical active ingredients and pharmaceutically acceptable excipients, such as carriers, diluents, adjuvants, colorants, etc.; for feeds, the product may contain main feed ingredients, such as soybean meal, hay, etc., and feed excipients acceptable to feeds, such as sweeteners, flavors, preservatives, fragrances, colorants, etc., but the present invention is not limited thereto.

The above-mentioned product is prepared by adding the cocrystal of lutein and adipic acid according to the present invention or the lutein composition according to the present invention. The product can be prepared according to the conventional process except adding the cocrystal of lutein and adipic acid according to the present invention or the lutein composition according to the present invention.

The present invention has been described in detail above, but the above embodiments are only illustrative in nature and are not intended to limit the present invention. In addition, this application is not limited by any theory described in the above prior art or the summary of the invention or the following examples.

Unless otherwise explicitly stated, the numerical ranges throughout the application document include any sub-ranges therein and any numerical values incremented by the smallest sub-unit of a given value therein. Unless otherwise explicitly stated, the numerical values throughout the application document represent approximate measurements or limitations of the given values with a slight deviation and the range of embodiments having approximately the mentioned values and having the mentioned exact values. Except for the working examples provided at the end of the detailed description, all numerical values of parameters (e.g., quantities or conditions) in this application document (including the attached claims) should be understood in all cases as modified by the term “about” or “approximately”, regardless of whether “about” or “approximately” actually appears before the numerical value or not. “About” or “approximately” means that the numerical value described allows for slight imprecision (some close to precision in the value; approximately or reasonably close to the value; approximately). If the imprecision provided by “approximately” is not understood in this ordinary meaning in the art, the “approximately” used herein at least represents the changes that can be produced by ordinary processes of measuring and using these parameters. For example, “about” or “approximately” may include changes of less than or equal to 10%, less than or equal to 5%, less than or equal to 4%, less than or equal to 3%, less than or equal to 2%, less than or equal to 1% or less than or equal to 0.5%, and in some aspects, changes of less than or equal to 0.1%.

Unless otherwise expressly stated, the terms “comprising”, “including”, “having”, “containing” or any other similar terms throughout the application document are open terms, which indicate that a cocrystal or product may include other elements that are not explicitly listed but are generally inherent to the cocrystal or product in addition to the elements listed herein. In addition, in this application, the interpretation of the terms “comprising”, “including”, “having”, and “containing” should be considered as specifically disclosed and simultaneously cover closed or semi-closed conjunctions such as “consisting of” and “substantially consisting of”. “Substantially consisting of” means that the elements listed herein account for 95% or more, 97% or more, or in some aspects, 99% or more of the cocrystal, product or composition.

Compared with the prior art, the beneficial effects of the present invention include the followings:

The present invention provides a stable cocrystal of lutein and adipic acid. Compared with lutein itself, the cocrystal has significantly improved chemical stability and a higher melting point. The preparation process of the cocrystal disclosed in the present invention is simple, low cost, environmentally friendly, easy to control and reproducible. In addition, the cocrystal disclosed in the present invention has more excellent chemical stability, greatly improves the convenience of the application of lutein, saves costs in storage, transportation and use, and can further broaden the application range of lutein. Therefore, the cocrystal of lutein and adipic acid has a strong practical application value.

### BRIEF DESCRIPTION OF THE DRAWING

FIG. 1 is an X-ray powder diffraction (XRPD) pattern of the cocrystal of lutein and adipic acid prepared in Example 1;

FIG. 2 is a differential scanning calorimetry (DSC) curve of the cocrystal of lutein and adipic acid prepared in Example 1;

FIG. 3 is an infrared spectrum (IR) diagram of the cocrystal of lutein and adipic acid prepared in Example 1;

FIG. 4 is an X-ray powder diffraction (XRPD) pattern of the mixture containing the cocrystal of lutein and adipic acid (lutein:adipic acid=1:2) prepared in Example 5;

FIG. 5 is an X-ray powder diffraction (XRPD) pattern of the mixture containing the cocrystal of lutein and adipic acid (lutein:adipic acid=2:1) prepared in Example 6;

FIG. 6 is an X-ray powder diffraction (XRPD) pattern of the mixture containing the cocrystal of lutein and adipic acid (lutein:adipic acid=1:5) prepared in Example 7;

FIG. 7 is an X-ray powder diffraction (XRPD) pattern of the mixture containing the cocrystal of lutein and adipic acid (lutein:adipic acid=5:1) prepared in Example 8;

FIG. 8 is an X-ray powder diffraction (XRPD) pattern of the mixture containing the cocrystal of lutein and adipic acid (lutein:adipic acid=1:10) prepared in Example 9;

FIG. 9 is an X-ray powder diffraction (XRPD) pattern of the mixture containing the cocrystal of lutein and adipic acid (lutein:adipic acid=10:1) prepared in Example 10.

### DETAILED DESCRIPTION

In order to make the purposes, technical solutions and advantages of the present invention clearer, the present invention is further described in detail in combination with the accompanying drawings and examples. It should be understood that the specific examples described herein are only used to explain the present invention and are not used to limit the present invention.

The X-ray powder diffraction pattern in the present invention was obtained by using a Bruker D8 Advanced X-ray diffractometer, which uses Cu—Kα irradiation (λ=1.54056 Å), the scanning range is from 3° to 40° of 2θ, and the scanning rate is 5°/minute.

The differential scanning calorimetry was performed on a TA DSC Q2000 instrument with a heating rate of 10 K/min.

The infrared spectrum (IR) was collected by a Fourier transform infrared spectrometer Thermo Scientific Nicolet 6700.

A Jingxin JX-2G planetary ball mill was used for ball milling.

An Agilent 1260 Infinity HPLC was used to determine the Lutein content.

Unless stated otherwise, the raw materials, reagents, equipment, methods, etc. used in this application are all conventional raw materials, reagents, equipment, methods in the field.

### Example 1

1.46 g of adipic acid and 5.69 g of lutein (molar ratio 1:1) were added to 60 ml of acetone as a solvent, stirred and suspended at 30° C. for 12 hours, filtered to obtain a yellow solid, which was vacuum dried overnight to obtain a cocrystal of lutein and adipic acid.

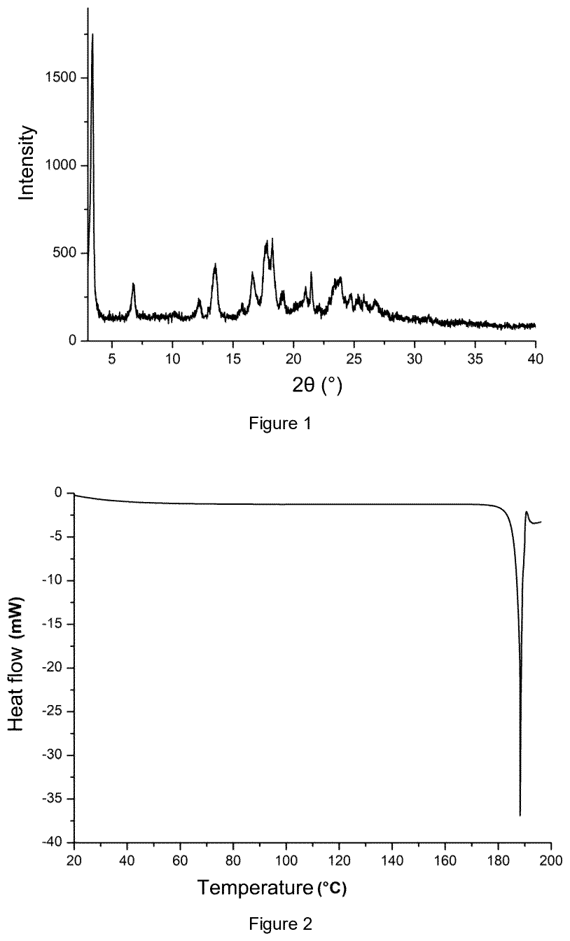

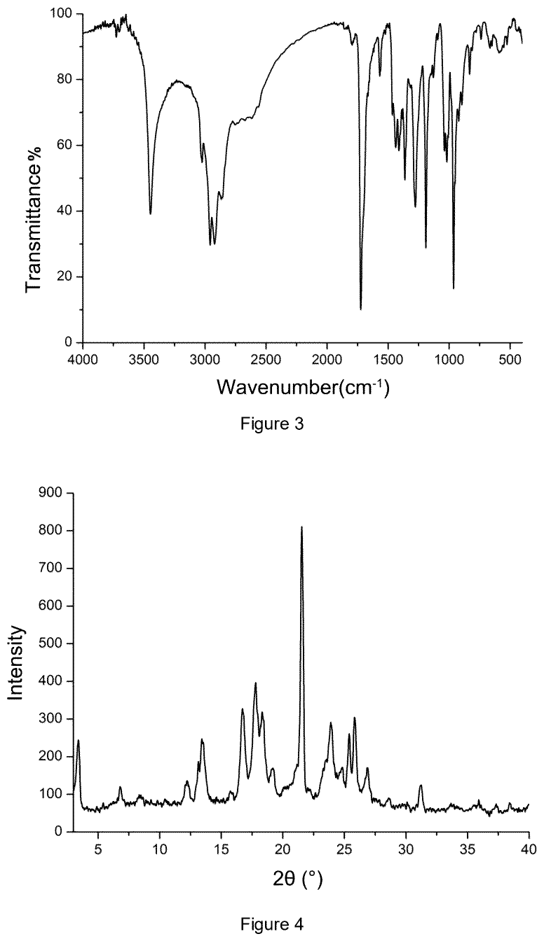

This cocrystal was characterized by solid-state methods such as X-ray powder diffraction (XRPD), differential scanning calorimetry (DSC) and infrared (IR) spectroscopy. The results are shown in FIGS. 1 to 3, respectively.

The peak values of the X-ray powder diffraction pattern of the cocrystal of lutein and adipic acid obtained as above are shown in Table 1:

TABLE 1
Peak positions and intensities

| Position (2θ, °) | Relative intensity (%) |
|---|---|
| 3.38 | 100 |
| 6.77 | 11.7 |
| 12.24 | 5.8 |
| 13.55 | 17.9 |
| 15.72 | 2.8 |
| 16.62 | 11.0 |
| 17.78 | 21.0 |
| 18.24 | 21.4 |
| 19.04 | 5.4 |
| 21.0 | 7.4 |
| 21.47 | 12.3 |
| 23.41 | 9.1 |
| 23.87 | 10.1 |
| 24.66 | 4.2 |
| 25.32 | 3.6 |
| 25.82 | 3.3 |
| 26.76 | 3.3 |

### Example 2

1.46 g of adipic acid and 5.69 g of lutein (molar ratio 1:1) were added to 60 ml of ethyl acetate as a solvent, stirred and suspended at 30° C. for 12 hours, filtered to obtain a yellow solid, which was vacuum dried overnight to obtain a cocrystal of lutein and adipic acid.

This cocrystal was characterized by solid-state methods such as X-ray powder diffraction (XRPD), differential scanning calorimetry (DSC) and infrared (IR) spectroscopy. The results are basically consistent with FIGS. 1-3.

### Example 3

0.146 g of adipic acid and 0.569 g of lutein (molar ratio 1:1) were added to a ball mill, 1 ml of acetone was added therein, and the mixture was ball milled for 1 hour, and then dried at room temperature in a vacuum drying oven for 12 hours to obtain a cocrystal of lutein and adipic acid.

This cocrystal was characterized by solid-state methods such as X-ray powder diffraction (XRPD), differential scanning calorimetry (DSC) and infrared (IR) spectroscopy. The results are basically consistent with FIGS. 1-3.

### Example 4

0.146 g of adipic acid and 0.569 g of lutein (molar ratio 1:1) were added to a ball mill, 1 ml of ethyl acetate was added therein, and the mixture was ball milled for 1 hour, and then dried in a vacuum oven at room temperature for 12 hours to obtain a cocrystal of lutein and adipic acid.

This cocrystal was characterized by solid-state methods such as X-ray powder diffraction (XRPD), differential scanning calorimetry (DSC) and infrared (IR) spectroscopy. The results are basically consistent with FIGS. 1-3.

### Example 5

0.292 g of adipic acid and 0.569 g of lutein (molar ratio 2:1) were added to a ball mill, 1 ml of acetone was added therein, and the mixture was ball milled for 1 hour, and then dried in a vacuum oven at room temperature for 12 hours to obtain a mixture containing the cocrystal of lutein and adipic acid.

The obtained mixture was characterized by X-ray powder diffraction (XRPD), and the results are shown in FIG. 4.

The peak values of the X-ray powder diffraction pattern of the obtained lutein composition with the stoichiometric ratio of lutein to adipic acid of 1:2 are shown in Table 2:

TABLE 2
Peak positions and intensities

| Position (2θ, °) | Relative intensity (%) |
|---|---|
| 3.40 | 21.0 |
| 6.79 | 7.2 |
| 12.21 | 8.0 |
| 13.13 | 14.1 |
| 13.45 | 22.9 |
| 15.74 | 3.3 |
| 16.74 | 28.8 |
| 17.80 | 36.2 |
| 18.32 | 24.9 |
| 19.20 | 6.8 |
| 21.55 | 100 |
| 23.89 | 23.3 |
| 25.38 | 19.5 |
| 28.82 | 26.6 |
| 26.85 | 10.7 |
| 31.23 | 9.8 |

### Example 6

0.146 g of adipic acid and 1.138 g of lutein (molar ratio 1:2) were added to a ball mill, 1 ml of acetone was added therein, and the mixture was ball milled for 1 hour, and then dried in a vacuum oven at room temperature for 12 hours to obtain a mixture containing a cocrystal of lutein and adipic acid.

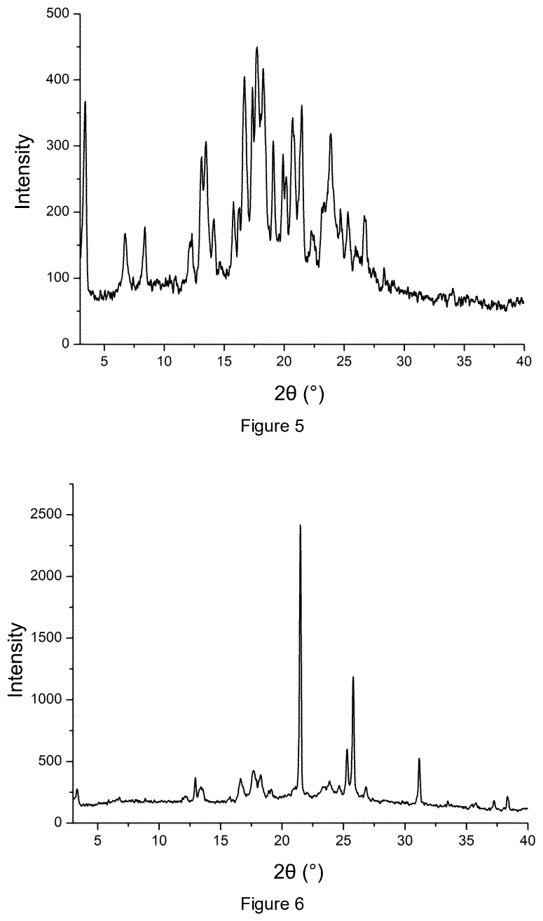

The obtained mixture was characterized by X-ray powder diffraction (XRPD), and the results are shown in FIG. 5.

The peaks of the X-ray powder diffraction pattern of the lutein composition obtained with a stoichiometric ratio of lutein to adipic acid of 2:1 as above are shown in Table 3:

TABLE 3
Peak positions and intensities

| Position (2θ, °) | Relative intensity (%) |
|---|---|
| 3.42 | 96.6 |
| 6.75 | 33.5 |
| 8.39 | 35.4 |
| 12.30 | 28.1 |
| 13.13 | 65.0 |
| 13.49 | 76.4 |
| 14.15 | 22.4 |
| 15.77 | 31.6 |
| 16.70 | 87.5 |
| 17.74 | 100 |
| 18.28 | 74.5 |
| 19.08 | 54.8 |
| 19.90 | 46.4 |
| 20.71 | 71.9 |
| 21.47 | 81.4 |
| 22.33 | 16.0 |
| 23.31 | 28.5 |
| 23.87 | 68.8 |
| 25.30 | 28.9 |
| 26.68 | 33.5 |

### Example 7

0.73 g of adipic acid and 0.569 g of lutein (molar ratio 5:1) were added to a ball mill, 1 ml of acetone was added therein, and the mixture was ball milled for 1 hour, and then dried in a vacuum oven at room temperature for 12 hours to obtain a mixture containing a cocrystal of lutein and adipic acid.

The obtained mixture was characterized by X-ray powder diffraction (XRPD), and the results are shown in FIG. 6.

In the figure, the peaks of the X-ray powder diffraction pattern of the lutein composition obtained with a stoichiometric ratio of lutein to adipic acid of 1:5 as above are shown in Table 4:

TABLE 4
Peak positions and intensities

| Position (2θ, °) | Relative intensity (%) |
|---|---|
| 3.32 | 4.0 |
| 12.13 | 2.1 |
| 12.95 | 8.5 |
| 13.47 | 5.0 |
| 15.77 | 2.0 |
| 16.64 | 7.3 |
| 17.68 | 9.4 |
| 18.26 | 7.9 |
| 19.12 | 3.1 |
| 20.95 | 2.6 |
| 21.49 | 100 |
| 23.37 | 2.8 |
| 23.87 | 5.0 |
| 24.66 | 3.0 |
| 25.30 | 16.8 |
| 25.80 | 43.8 |
| 26.82 | 3.9 |
| 31.15 | 17.2 |
| 33.50 | 2.0 |
| 35.77 | 2.5 |
| 37.25 | 3.5 |
| 38.35 | 5.3 |

### Example 8

0.146 g of adipic acid and 2.845 g of lutein (molar ratio 1:5) were added to a ball mill, 1 ml of acetone was added therein, and the mixture was ball milled for 1 hour, and then dried in a vacuum oven at room temperature for 12 hours to obtain a mixture containing a cocrystal of lutein and adipic acid.

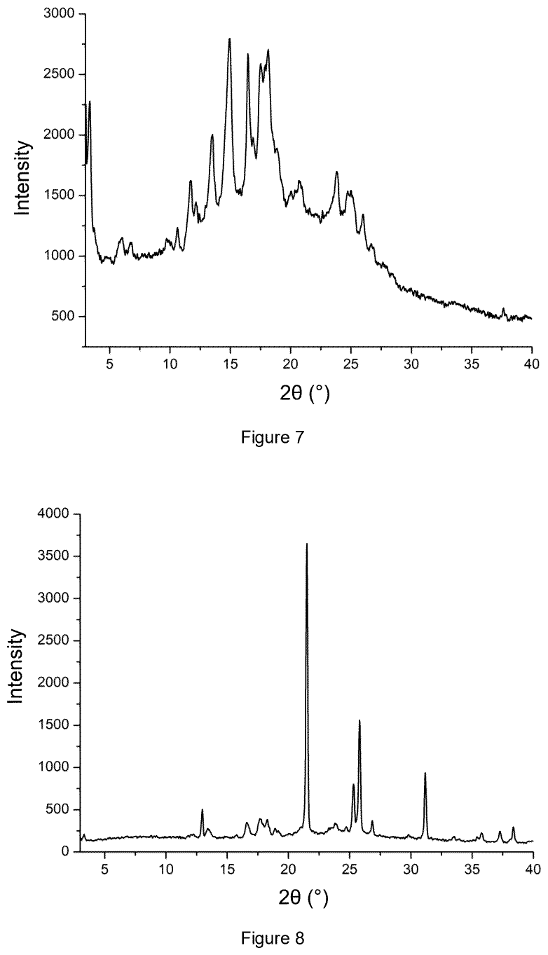

The obtained mixture was characterized by X-ray powder diffraction (XRPD), and the results are shown in FIG. 7.

The peak values of the X-ray powder diffraction pattern of the lutein composition obtained with a stoichiometric ratio of lutein to adipic acid of 5:1 as above are shown in Table 5:

TABLE 5
Peak positions and intensities

| Position (2θ, °) | Relative intensity (%) |
|---|---|
| 3.36 | 44.3 |
| 6.01 | 13.9 |
| 6.71 | 8.1 |
| 10.62 | 13.9 |
| 11.68 | 33.6 |
| 12.16 | 12.9 |
| 13.53 | 46.7 |
| 14.97 | 100 |
| 15.79 | 3.0 |
| 16.46 | 77.1 |
| 17.52 | 54.4 |
| 18.14 | 73.1 |
| 20.79 | 17.0 |
| 23.81 | 26.0 |
| 25.12 | 18.7 |
| 26.00 | 15.7 |

### Example 9

1.46 g of adipic acid and 0.569 g of lutein (molar ratio 10:1) were added to a ball mill, 1 ml of acetone was added, and the mixture was ball milled for 1 hour, and then dried in a vacuum oven at room temperature for 12 hours to obtain a mixture containing a cocrystal of lutein and adipic acid.

The obtained mixture was characterized by X-ray powder diffraction (XRPD), and the results are shown in FIG. 8.

The peak values of the X-ray powder diffraction pattern of the lutein composition obtained with a stoichiometric ratio of lutein to adipic acid of 1:10 as above are shown in Table 6:

TABLE 6
Peak positions and intensities

| Position (2θ, °) | Relative intensity (%) |
|---|---|
| 3.32 | 1.9 |
| 12.04 | 1.2 |
| 12.97 | 9.6 |
| 13.47 | 3.3 |
| 15.77 | 1.1 |
| 16.60 | 4.9 |
| 17.67 | 5.6 |
| 18.26 | 5.2 |
| 18.92 | 2.3 |
| 20.97 | 2.2 |
| 21.51 | 100 |
| 23.29 | 1.8 |
| 23.83 | 2.8 |
| 24.74 | 1.5 |
| 25.32 | 16.4 |
| 25.83 | 38.8 |
| 26.84 | 4.8 |
| 29.83 | 1.3 |
| 31.17 | 23.0 |
| 33.54 | 1.7 |
| 25.40 | 1.7 |
| 35.77 | 3.1 |
| 37.27 | 3.8 |
| 38.37 | 5.5 |

### Example 10

0.146 g of adipic acid and 5.69 g of lutein (molar ratio 1:10) were added to a ball mill, 2 ml of acetone was added, and the mixture was ball milled for 1 hour, and then dried in a vacuum oven at room temperature for 12 hours to obtain a mixture containing a cocrystal of lutein and adipic acid.

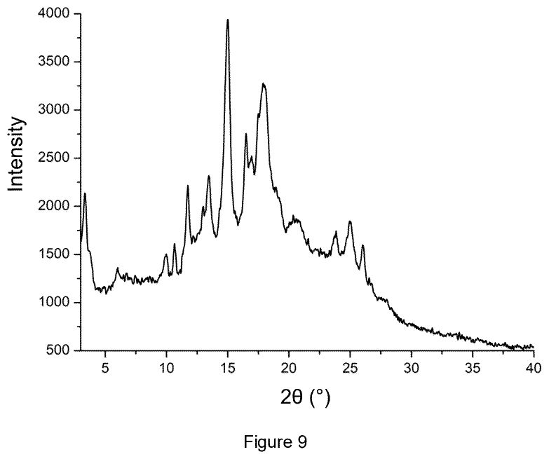

The obtained mixture was characterized by X-ray powder diffraction (XRPD), and the results are shown in FIG. 9.

The peaks of the X-ray powder diffraction pattern of the lutein composition obtained with a stoichiometric ratio of lutein to adipic acid of 10:1 as above are shown in Table 7:

TABLE 7
List of the peaks

| Position (2θ, °) | Relative intensity (%) |
|---|---|
| 3.34 | 29.2 |
| 6.02 | 7.7 |
| 10.00 | 10.5 |
| 10.66 | 14.3 |
| 11.74 | 31.1 |
| 12.18 | 4.0 |
| 13.00 | 15.4 |
| 13.47 | 29.2 |
| 15.01 | 100 |
| 15.59 | 1.1 |
| 16.50 | 30.9 |
| 16.95 | 11.9 |
| 17.50 | 27.9 |
| 18.02 | 46.2 |
| 20.38 | 9.5 |
| 20.77 | 10.0 |
| 23.85 | 11.4 |
| 24.96 | 19.1 |
| 26.02 | 13.8 |

### Experimental Example (Comparison of the Stability of Lutein Itself and the Lutein Cocrystals According to the Present Invention)

Representative studies were conducted using commercially available lutein crystals (purchased from Chenguang Biotechnology) and cocrystals containing lutein and adipic acid obtained in Examples 1 and 3. It can be confirmed that the lutein composition described in the context has significantly improved stability compared to lutein itself.

A stability comparison was made among the commercially available lutein, the cocrystals of lutein and adipic acid obtained in Examples 1 and 3, and the mixtures containing the cocrystal of lutein and adipic acid obtained in Examples 5-10. The commercially available lutein crystal, the cocrystals of lutein and adipic acid obtained in Examples 1 and 3, and the mixtures containing the cocrystal of lutein and adipic acid obtained in Examples 5-10 were stored in an open state and away from light under a condition of 40° C./75% relative humidity. The lutein content was analyzed by HPLC. The results are shown in Table 8.

TABLE 8
Stability results of lutein under accelerated conditions
(40° C./75% relative humidity, open state)

| | Day 0 | Day 3 | Day 5 | Day 7 |
|---|---|---|---|---|
| Commercially available lutein | 100.0% | 75.4% | 55.2% | 38.1% |
| crystal | | | | |
| Lutein cocrystal (Example 1) | 100.0% | 101.1% | 100.3% | 97.7% |
| Lutein cocrystal (Example 3) | 100.0% | 99.5% | 98.7% | 97.0% |
| Composition containing lutein | 100.0% | 96.8% | 93.8% | 90.8% |
| cocrystal (Example 5) | | | | |
| Composition containing lutein | 100.0% | 90.8% | 85.3% | 79.7% |
| cocrystal (Example 6) | | | | |
| Composition containing lutein | 100.0% | 97.3% | 95.2% | 89.0% |
| cocrystal (Example 7) | | | | |
| Composition containing lutein | 100.0% | 98.4% | 97.2% | 93.5% |
| cocrystal (Example 8) | | | | |
| Composition containing lutein | 100.0% | 96.7% | 93.8% | 88.1% |
| cocrystal (Example 9) | | | | |
| Composition containing lutein | 100.0% | 96.3% | 94.5% | 91.5% |
| cocrystal (Example 10) | | | | |

As shown in the above results, as compared with commercially available lutein crystal, the lutein cocrystal disclosed in the present invention has more excellent stability and can remain stable for a long time under high temperature and high humidity conditions without taking special protective measures against oxygen. The stability of the composition containing lutein cocrystal is slightly worse than that of the present lutein cocrystal, but it is still much better than the stability of commercially available lutein crystal.

### Comparative Example 1

0.118 g of succinic acid and 0.569 g of lutein were added to a ball mill, 1 ml of acetone or ethyl acetate was added, and the mixture was ball milled for 1 hour, and then dried at room temperature in a vacuum oven for 12 hours to obtain a yellow powder.

This powder was detected by X-ray powder diffraction (XRPD) and found that no cocrystal was formed.

### Comparative Example 2

0.132 g of glutaric acid and 0.569 g of lutein were added to a ball mill, 1 ml of acetone or ethyl acetate was added, and the mixture was ball milled for 1 hour, and then dried at room temperature in a vacuum oven for 12 hours to obtain a yellow powder.

This powder was detected by X-ray powder diffraction (XRPD) and found that no cocrystal was formed.

### Comparative Example 3

0.160 g of pimelic acid and 0.569 g of lutein were added to a ball mill, 1 ml of acetone or ethyl acetate was added, and the mixture was ball milled for 1 hour, and then dried in a vacuum oven at room temperature for 12 hours to obtain a yellow powder.

This powder was detected by X-ray powder diffraction (XRPD) and found that no cocrystal was formed.

### Comparative Example 4

0.174 g of suberic acid and 0.569 g of lutein were added to a ball mill, 1 ml of acetone or ethyl acetate was added, and the mixture was ball milled for 1 hour, and then dried in a vacuum oven at room temperature for 12 hours to obtain a yellow powder.

This powder was detected by X-ray powder diffraction (XRPD) and found that no cocrystal was formed.

It can be seen that when succinic acid, glutaric acid, pimelic acid, and suberic acid similar to adipic acid are used, they cannot form cocrystal with lutein and cannot improve the stability of lutein thereby.

1. A cocrystal of lutein and adipic acid, characterized in that the stoichiometric ratio of lutein to adipic acid in the cocrystal is 1:1.

2. The cocrystal of lutein and adipic acid according to claim 1, having an X-ray powder diffraction pattern comprising characteristic peaks at 2θ of 3.4°±0.2°, 12.2°±0.2°, 13.6°±0.2°, 15.7°±0.2°, 16.6°±0.2°, and 17.8°±0.2°.

3. The cocrystal of lutein and adipic acid according to claim 1, having a differential scanning calorimetry analysis curve comprising a characteristic endothermic peak at about 187±2° C., when the temperature is increased at a rate of 10° C./min as measured by differential scanning calorimetry.

4. The cocrystal of lutein and adipic acid according to claim 1, having an infrared spectrum comprising characteristic peaks at 3446 cm⁻¹, 1723 cm⁻¹, and 963 cm⁻¹.

5. A process for preparing the cocrystal of lutein and adipic acid according to claim 1, wherein the process is one of the following processes:
Process 1:
recrystallizing lutein and adipic acid in a molar ratio of 1:1 in a solvent, and drying the resultant precipitate to obtain the cocrystal of lutein and adipic acid; or
Process 2:
ball-milling lutein and adipic acid in a molar ratio of 1:1 in a solvent for 10 minutes or more, and then drying the obtained solid to obtain the cocrystal of lutein and adipic acid.

6. The process according to claim 5, wherein,
in process 1, lutein and adipic acid are stirred and suspended in a solvent at 10-40° C. for 5-30 hours, and the resultant precipitate is dried to obtain the cocrystal of lutein and adipic acid;
in process 2, lutein and adipic acid are ball-milled in a solvent for 30 minutes to two hours, and then the obtained solid is dried to obtain the cocrystal of lutein and adipic acid.

7. A lutein composition comprising the cocrystal of lutein and adipic acid according to claim 1.

8. The lutein composition according to claim 7, wherein the stoichiometric ratio of lutein to adipic acid in the lutein composition is 10:1 to 1:10.

9. A lutein product, comprising the cocrystal of lutein and adipic acid according to claim 1, wherein the product is selected from health products, foods, cosmetics, medicines, pharmaceutical excipients and feeds.

10. (canceled)

11. The cocrystal of lutein and adipic acid according to claim 1, having an X-ray powder diffraction pattern comprising characteristic peaks at 2θ of 3.4°±0.2°, 12.2°±0.2°, 13.6°±0.2°, 15.7°±0.2°, 16.6°±0.2°, 17.8°±0.2°, 18.2±0.2°, and 21.5±0.2°.

12. The cocrystal of lutein and adipic acid according to claim 1, characterized by an X-ray powder diffraction pattern substantially as shown in FIG. 1.

13. The process according to claim 5, wherein the solvent is one or more selected from the group consisting of water, alcohols, ketones, esters, alkanes, aromatic hydrocarbons and halogenated alkanes.

14. The lutein composition according to claim 7, wherein the stoichiometric ratio of lutein to adipic acid in the lutein composition is 1:2.

15. The lutein composition according to claim 7, wherein the stoichiometric ratio of lutein to adipic acid in the lutein composition is 2:1.

16. The lutein composition according to claim 7, wherein the stoichiometric ratio of lutein to adipic acid in the lutein composition is 1:5.

17. The lutein composition according to claim 7, wherein the stoichiometric ratio of lutein to adipic acid in the lutein composition is 5:1.

18. The lutein composition according to claim 7, wherein the stoichiometric ratio of lutein to adipic acid in the lutein composition is 1:10.

19. The lutein composition according to claim 7, wherein the stoichiometric ratio of lutein to adipic acid in the lutein composition is 10:1.

20. A lutein product, comprising the lutein composition according to claim 7, wherein the product is selected from health products, foods, cosmetics, medicines, pharmaceutical excipients and feeds.
